(19) United States
(12) Patent Application Publication
(10) Pub. No.: US 2025/0363914 A1
(43) Pub. Date: Nov. 27, 2025

(54) BIONIC ORGAN DEVICE

(71) Applicant: DARWIN PRECISIONS CORPORATION, Hsinchu County (TW)

(72) Inventors: Ting-Chieh Yang, Hsinchu County (TW); Chung-Yi Huang, Hsinchu County (TW)

(21) Appl. No.: 19/026,533

(22) Filed: Jan. 17, 2025

(30) Foreign Application Priority Data
May 24, 2024 (TW) 113119390

(51) Int. Cl. G09B 23/28

(52) U.S. Cl. CPC G09B 23/28

(57) ABSTRACT

A bionic organ device comprising an organ chip and a magnetic field generating module. The organ chip comprises a first body, a second body, and a magnetically-driven porous membrane. The magnetically-driven porous membrane is disposed between the first body and the second body and forms a channel system with the first body and the second body. The magnetically-driven porous membrane comprises a magnetic material and a membrane body, and the magnetic material is disposed in the membrane body. The magnetic field generating module is disposed outside the organ chip and is adapted to generate a magnetic field, the magnetic field passes through the magnetically-driven porous membrane and cause the magnetically-driven to stretch, deform, or a combination thereof in response to a magnetic force in the magnetic field.

### FIELD OF THE INVENTION

The present invention relates to a bionic technology, and more particularly to a bionic organ device that can simulate the microenvironment in an organism.

### BACKGROUND OF THE INVENTION

Traditional cell culture models cannot reflect the complicated physiological functions of tissues and organs in an organism. Animal experiments have drawbacks such as long period and high cost. Organ chips reconstruct the in vivo physiological environment of organs, simulating the structures, microenvironment, and physiological functions of organs in the organism. Organ chips also offer accurate parameter control, along with advantages of miniaturization, integration, high efficiency, and reduced cost. To simulate the dynamic microenvironment in the organism or the stretching and contraction of cells, current organ chips are equipped with the vacuum system that achieve bionic effects through vacuum application. However, the vacuum system which carries out the stretching also pulls on the membrane to which cells are attached, potentially causing membrane damage and malfunction of the organ chips. In addition, the manufacturing of the vacuum system is complicated and improvement is therefore required.

### SUMMARY OF THE INVENTION

The invention provides a bionic organ device which can be used to simulate dynamic microenvironment of organs, and has a more simplified structure which is beneficial to have a streamlined manufacturing process, to reduce cost, and to improve yield.

The bionic organ device provided by the invention comprises an organ chip and a magnetic field generating module. The organ chip comprises a first body, a second body, and a magnetically-driven porous membrane. The magnetically-driven porous membrane is disposed between the first body and the second body and forms a channel system with the first body and the second body. The magnetically-driven porous membrane further comprises a magnetic material and a membrane body, and the magnetic material is disposed in the membrane body. The magnetic field generating module is disposed outside the organ chip and is adapted to generate a magnetic field, the magnetic field passes through the magnetically-driven porous membrane and cause the magnetically-driven to stretch, deform, or a combination thereof in response to a magnetic force in the magnetic field.

The present invention utilizes a magnetically-driven porous membrane and a magnetic field generating module, enabling the simulation of the dynamic microenvironment in the organism and the performances of organs, tissues, or cells in such dynamic microenvironment, and making it more convenient to use. Further, the organ chip of the present invention has a simplified structure, which is beneficial to have a more streamlined manufacturing process, to reduce cost, and to improve yield.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic three-dimensional view of a bionic organ device according to an embodiment of the invention;

FIG. 2 is a partial schematic three-dimensional exploded view of a bionic organ device according to an embodiment of the invention;

FIG. 3 is a schematic cross-sectional view taken along line A-A″ in FIG. 1;

FIG. 4 is a schematic cross-sectional view of a magnetically-driven porous membrane according to an embodiment of the invention;

FIG. 5 is a schematic view of an operation of a bionic organ device according to an embodiment of the invention; and

FIG. 6 is a schematic view of an operation of a bionic organ device accordingly to another embodiment of the invention.

### DETAILED DESCRIPTION OF PREFERRED EMBODIMENTS

The foregoing and other technical contents and other features and advantages of the present invention will be clearly presented from the following detailed description of a preferred embodiment in cooperation with the accompanying drawings. Directional terms mentioned in the following examples are only used to describe directions referring to the attached drawings. Therefore, the directional terms used are for illustration and not for limitation. In addition, terms such as “first” and “second” involved in the description or claims are merely used for naming the elements or distinguishing different embodiments or ranges rather than limiting the upper limit or lower limit of the quantity of the elements.

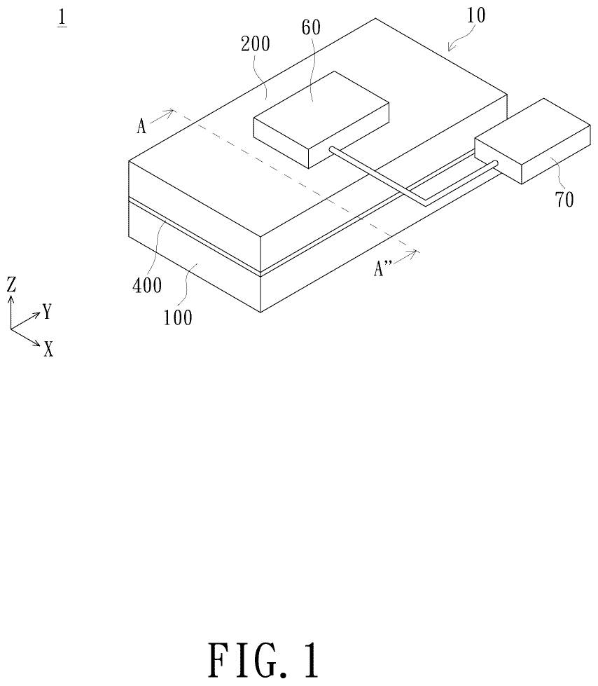

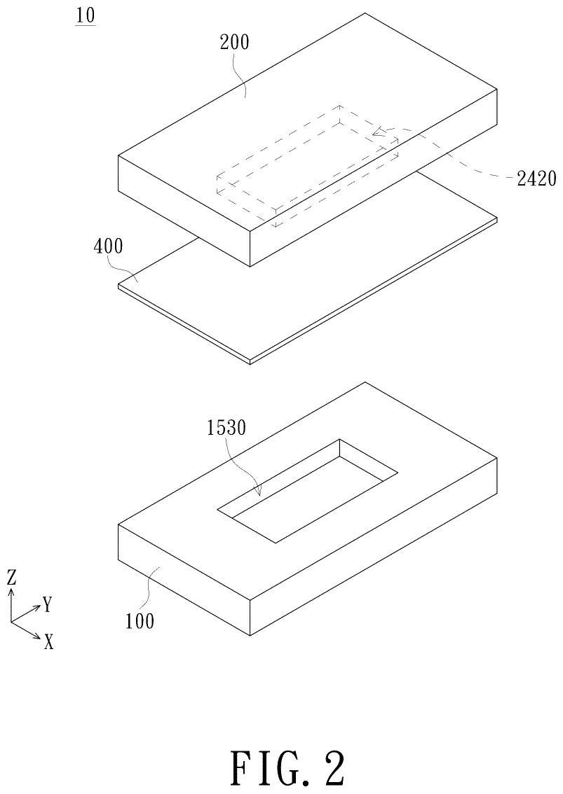

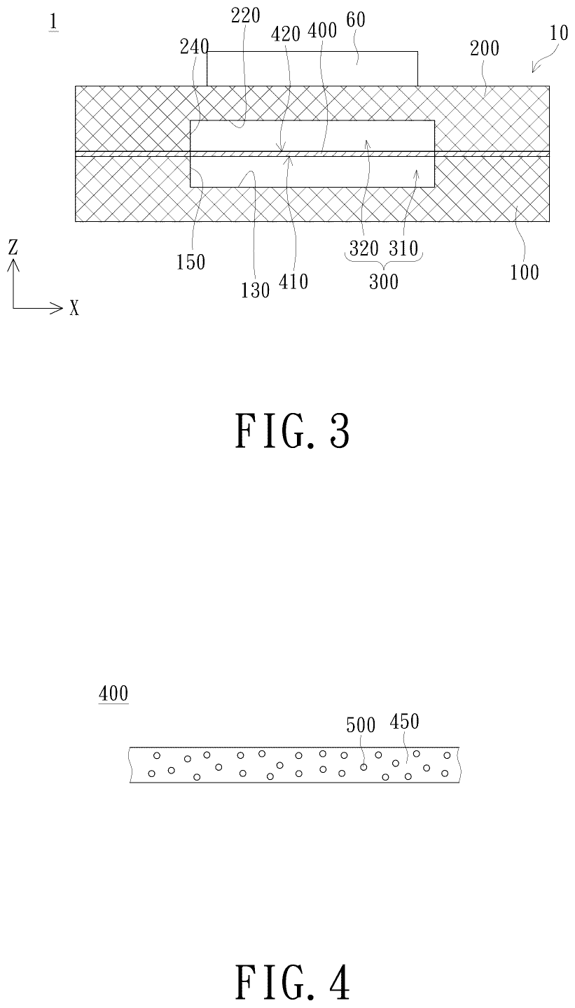

FIG. 1 is a schematic three-dimensional view of a bionic organ device according to an embodiment of the invention, FIG. 2 is an exploded view of FIG. 1, and FIG. 3 is a schematic cross-sectional view taken along line A-A″ in FIG. 1. As shown in FIGS. 1 to 3, the bionic organ device of the embodiment of the present invention includes an organ chip 10 and a magnetic field generating module 60. The magnetic field generating module 60 is disposed outside the organ chip 10 and is adapted to generate a magnetic field (described in detail later), allowing the organ chip 10 to further simulate such as the dynamic microenvironment of organs, tissues, or cells in an organism. The magnetic field generating module 60 may be disposed close to the organ chip 10, such as on the organ chip 10, but is not limited thereto. The relative position between the magnetic field generation module 60 and the organ chip 10 should primarily ensure that the organ chip is within the influence range of the magnetic field.

The organ chip 10 includes a first body 100, a second body 200, and a magnetically-driven porous membrane 400. The magnetically-driven porous membrane 400 is disposed between the first body 100 and the second body 200 and forms a channel system 300 with the first body 100 and the second body 200. FIGS. 1 to 3 illustrate primarily the positional relationship among the first body 100, the second body 200, the channel system 300, and the magnetically-driven porous membrane 400. It is understood that the relative sizes of these four components are not necessarily as depicted in the figures. Further, the magnetically-driven porous membrane 400 is within the influence range of the magnetic field and can stretch and deform in response to magnetic force.

As shown in FIGS. 2 and 3, the first body 100 and the second body 200 each may have a groove-like structure with an accommodating space and an opening. In the embodiment of the present invention, the first body 100 may include a bottom portion 130 and a wall portion 150. The wall portion 150 is formed along the perimeter of the bottom portion 130, and the bottom portion 130 and the wall portion 150 together form an accommodating space 1530 within the first body 100. The structure of the second body 200 may be the same as or similar to that of the first body 100. In the embodiment of the present invention, the second body 200 may include a bottom portion 220 and a wall portion 240 formed along the perimeter of the bottom portion 220. The bottom portion 220 and the wall portion 240 together form an accommodating space 2420 within the second body 200. In the embodiment of the present invention, the first body 100 and the second body 200 are combined with their accommodating spaces 1530 and 2420 facing each other, where the magnetically-driven porous membrane 400 may be connected to the surface of the first body 100 facing the second body 200 and the surface of the second body 200 facing the first body 100. The connecting among the driven porous membrane 400, the first body 100, and the second body 200 may be achieved by one or more known methods such as thermal pressing, welding, adhesive bonding, or other means capable of achieving the connection, depending on the materials of the first body 100, the second body 200, and the magnetically-driven porous membrane 400.

The materials of the first body 100 and the second body 200 may be plastics, such as but not limited to, polycarbonate (PC), polyethylene terephthalate (PET), polypropylene (PP), polyvinyl chloride (PVC), and polydimethylsiloxane (PDMS). The magnetically-driven porous membrane 400 can include magnetic material 500 and a membrane body 450. As shown in FIG. 4, the magnetic material 500 may be disposed in the membrane body 450. The magnetic material 500 is further classified as a strong magnetic material, including ferromagnetic and ferrimagnetic materials, such as but not limited to, iron, cobalt, nickel, or their alloys and compounds. In addition, the magnetic material 500 can exhibit varying degrees of saturation magnetization and dispersibility, where high saturation magnetization indicates a stronger response to magnetic force, and high dispersibility helps to prevent aggregation. In the embodiment of the present invention, the magnetic material 500 is preferably ferromagnetic or ferrimagnetic material such as iron, cobalt, nickel, or their alloys and compounds, possessing suitable saturation magnetization and dispersibility and distributed in particle form within the membrane body 450. FIG. 4 illustrates mainly the positional relationship between the particles of the magnetic material 500 and the membrane body 450. It is understood that the relative sizes between the two are not necessarily as depicted in the figure. The particles of the magnetic material 500 can have a particle size in micrometers (μm), such as several micrometers, tens of micrometers, or dozens of micrometers.

In the embodiment of the present invention, the membrane body 450 can be made from natural materials or synthetic polymer materials. The synthetic polymer materials include, but are not limited to, polyethylene terephthalate (PETE), polydimethylsiloxane (PDMS), polyurethane, styrene-ethylene-butylene-styrene (SEBS), poly(hydroxyethyl methacrylate) (pHEMA), polyethylene glycol, polyvinyl alcohol, or polycarbonate (PC). The fabricated membrane body 450 exhibits compliant and porous, with pore diameters on the nanometer (nm) scale, such as dozens, tens, or hundreds of nanometers. Due to the compliance of the membrane body 450, the magnetically-driven porous membrane 400 is endowed with compliance, flexibility, and deformability. Due to the porosity of the membrane body 450, fluids or certain substances are able to move through the magnetically-driven porous membrane 400 between its two sides. In a preferred embodiment of the present invention, the membrane body 450 is made from hydrophilic polymer materials. These hydrophilic polymer materials may contain functional groups such as —OH, —CONH, —CONH₂, —COOH, and —SO₃H and can be formed by chemically or physically crosslinking monomers that possess these functional groups.

In a preferred embodiment of the present invention, the hydrophilic polymer materials include hydrogel. The hydrogel, for example, can be obtained by crosslinking of acrylic acid or its derivatives, acrylamide or its derivatives, and/or hydroxyethyl methacrylate or its derivatives, but are not limited thereto. When using hydrogel as the material for membrane body 450, for example, a magnetic powder 500 can be added and thoroughly mixed into the hydrogel in a sol or fluid state, and then modeled to form the magnetically-driven porous membrane 400, which includes the membrane body 450 and the magnetic powder 500. The mixing ratio of the magnetic powder 500 to the hydrogel can be, for example, in a weight ratio 0.5:1 to 4:1, such as 1:1, 1.5:1, 2:1, 2.5:1, 3:1, or 3.5:1. The modeling process may involve changes in temperature, such as heating or cooling, but is not limited thereto. In some embodiments of the invention, the magnetic powder 500 includes carbonyl iron powder. The carbonyl iron powder can have an appropriate size, for example, with a particle diameter of 5 to 9 μm.

The magnetically-driven porous membrane 400 further forms a first channel 310 of the channel system 300 with the first body 100 and forms a second channel 320 of the channel system 300 with the second body 200. The first channel 310 and/or the second channel 320 is able to allow at least one fluid (not shown) to pass through or remain within them. The magnetically-driven porous membrane 400 has a first membrane surface 410 and a second membrane surface 420, with the first membrane surface 410 located in the first channel 310 and the second membrane surface 420 located in the second channel 320. The magnetically-driven porous membrane 400 is adapted to be as a cell-attaching membrane. When used as a cell-attaching membrane, the first membrane surface 410 and the second membrane surface 420 can be used for cell attachment, and the first channel 310 and/or the second channel 320 may have fluid contacting cells on the first membrane surface 410 and/or the second membrane surface 420. For example, the first membrane surface 410 can be used for the attachment of the alveolar epithelial cells, while the second membrane surface 420 can be used for the attachment of the microvascular endothelial cells. The first channel 310 is supplied with an oxygen-rich gas, and the second channel 320 is supplied with a culture medium.

As described above, due to the porosity of the magnetically-driven porous membrane 400, certain molecules have the opportunity to pass through the magnetically-driven porous membrane 400 and move between the first channel 310 and the second channel 320. Accordingly, the organ chip 10 can simulate the dynamic microenvironment phenomena of organs, tissues, or cells within a biological organism. For example, the organ chip 10 can simulate the phenomena of the lung tissue cells in the microenvironment in the organism by having oxygen and carbon dioxide pass through the magnetically-driven porous membrane 400 respectively. The channel system 300 may also include input/output ports (not shown), which can be arranged in any known manner on the organ chip 10, further enabling e intercommunication between the interior and exterior of the organ chip 10.

As described above, the magnetically-driven porous membrane 400 has compliance and deformability, allowing it to respond to the magnetic force generated by the magnetic field of the magnetic field generating module 60. Further speaking, the magnetic force can attract or repel the magnetic material 500 in the magnetically-driven porous membrane 400, and preferably, the magnetic attraction or repulsion can drive the magnetically-driven porous membrane 400 to stretch and deform duo to the compliance of the membrane body 450. For example, when the direction of the magnetic attraction or magnetic repulsion is toward the first body 100, the magnetically-driven porous membrane 400 can bulge or stretch toward the first body 100 or the first channel 310. Additionally, when there is a higher content of the magnetic material 500 or a higher compliance of the membrane body 450, the same magnetic attraction or magnetic repulsion may cause a larger amount of deformation or greater extent of stretching. In some embodiments of the present invention, the magnetically-driven porous membrane 400 may bulge or stretch toward the first channel 310 and further compress the first channel 310. On the contrary, when the magnetically-driven porous membrane 400 bulges or stretches toward the second channel 320, it can further compress the second channel 320. In some embodiments of the present invention, the compression on the channels by the magnetically-driven porous membrane 400 may cause the pressure in the channel to increase.

In the embodiments of the present invention, the magnetic field generating module 60 is preferably powered by electricity to generate the magnetic field. As shown in FIG. 1, the bionic organ device 1 may further include a power supply 70, which is electrically connected to the magnetic field generating module 60 and to power the magnetic field generating module 60. Preferably, the magnetic field generating module 60 may include an electromagnet, and the power supply 70 can supply electric current to induce the electromagnet in the magnetic field generating module 60 to produce a magnetic field through electromagnetic induction. The magnetic field generating module 60 may further include an electromagnet capable of generating a magnetic force ranging from 0 to 30 Kg and a driving voltage between 12V to 110V. In some embodiments of the present invention, the magnetic field generating module 60 may be, for example, an electromagnet that capable of generating a magnetic force of, for example, 10 Kg, with a driving voltage of, for example, 12V.

As described above, the organ chip 10 is within the influence range of the magnetic field generated by the magnetic field generating module 60. The magnetic field generating module 60 can be disposed on the organ chip 10. In some embodiments of the present invention, as shown in FIGS. 1 to 3, the magnetic field generating module 60 is disposed on the second body 200 and close to the magnetically-driven porous membrane 400. Alternatively, the magnetic field generating module 60 is disposed on the first body 100. The specific location of the magnetic field generating module 60 on the first body 100 or the second body 200 can be further determined based on factors such as the direction of the magnetic field, the magnetic field strength, the magnitude of the magnetic force, and the magnetic flux density.

In a preferred embodiment of the present invention, the bionic organ device 1 may further include a control module (not shown). The control module may include components such as a microprocessor, microcontroller, or microcomputer, and is electrically connected to the magnetic field generating module 60, the power module 70, or a combination thereof. The electrical connection can be achieved through various known means, which may configurations such as circuit boards, adapters, etc., but are not limited thereto. The control module may be loaded with a control program that output a control signal to turn the power module 70 and the magnetic field generating module 60 on or off and to generate the magnetic field. The control module can further control the magnetic field strength, the magnitude of the magnetic force, the magnetic flux density, or a combination thereof. In some embodiments of the present invention, the control module may also control the direction of the magnetic field.

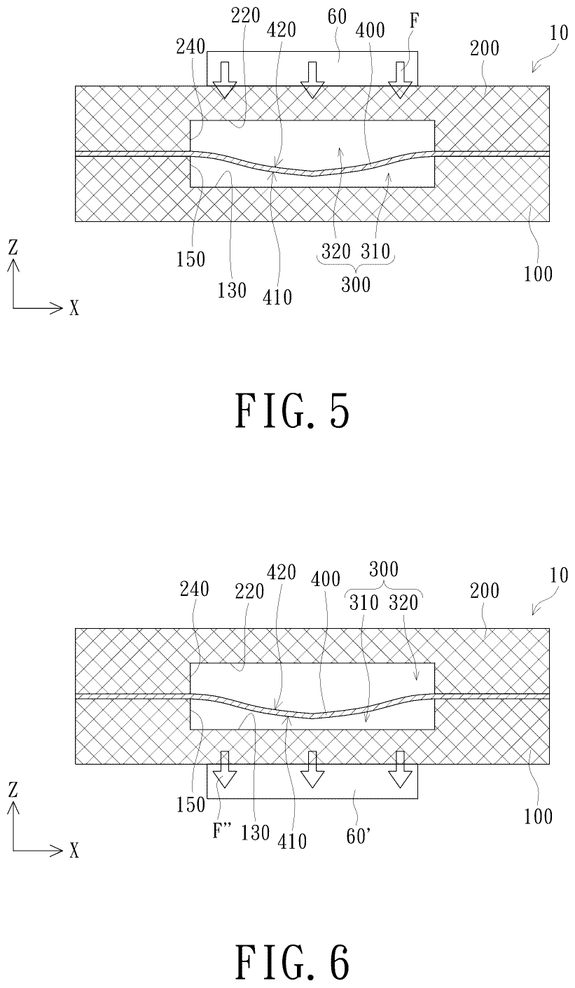

The operation of the bionic organ device 1 is illustrated with reference to the embodiments shown in FIGS. 1 to 3 and FIG. 5. As shown in FIG. 5, in some embodiments of the present invention, when the power supply 70 supplies power to the magnetic field generating module 60, the generated magnetic field can exert a magnetic repulsion force F on the magnetically-driven porous membrane 400, preferably, driving the magnetically-driven porous membrane to stretch. When the magnetic force is released, the magnetically-driven porous membrane 400 can return to its original state. Moreover, the magnetically-driven porous membrane 400 may stretch periodically. The periodic stretching of the magnetically-driven porous membrane 400 can be achieved, for example, by periodically supplying power from the power supply 70 to generate the magnetic repulsion force F periodically on the magnetically-driven porous membrane 400. When the magnetically-driven porous membrane 400 stretches periodically, it creates an effect similar to reciprocating motion.

In some embodiments of the present invention, when the magnetically-driven porous membrane 400 stretches in a direction such as toward the first channel 310 due to the magnetic repulsion force F, the magnetically-driven porous membrane 400 can further compress the first channel 310 thereby increasing the pressure in the first channel 310 and creating a pressure difference between the first channel 310 and the second channel 320. The pressure difference may be used to drive the movement of the fluids in the channel system 300 and the movement of certain substances between the two sides of the magnetically-driven porous membrane 400. On the other hand, in some embodiments, when the magnetically-driven porous membrane 400 stretches, it can also achieve the effect of stretching cells. Further, when the magnetically-driven porous membrane 400 stretches periodically and moves back and forth, for example, it can further simulate the dynamic performance of cells.

The magnetic force acting on the magnetically-driven porous membrane 400 is not limited to the magnetic repulsion force F. For example, in the embodiment shown in FIG. 5, the magnetic field generating module 60 may also generate a magnetic attraction force (not shown) on the magnetically-driven porous membrane 400, where the direction of the magnetic attraction force is opposite to the direction of the magnetic repulsion force F. In other words, the magnetic field generating module 60 can exert forces in two opposite directions on the magnetically-driven porous membrane 400, and either force can achieve the effect of stretching of the magnetically-driven porous membrane 400.

FIG. 6 shows another embodiment of the present invention. The difference from the embodiment shown in FIG. 5 is that the magnetic field generating module 60′ is disposed on the first body 100, and the generated magnetic field exerts a magnetic attraction force F″ on the magnetically-driven porous membrane 400 when the power supply 70 powers the magnetic field generating module 60′. Since the magnetic field generating module 60′ is disposed on the first body 100 which is relative to the second body 200, the direction of the magnetic attraction force F″ which acts on the magnetically-driven porous membrane 400 in the embodiment shown in FIG. 6 may be the same as the direction of the magnetic repulsion force F in the embodiment shown in FIG. 5. That is, different embodiments can achieve the effect of stretching the magnetically-driven porous membrane 400 with forces in the same direction. In some embodiments of the present invention, the magnetic repulsion force F or the magnetic attraction force F″ can be, for example, 0 to 100 mT, and preferably less than or equal to 70 mT. Further, when the magnetically-driven porous membrane 400 stretches and the deformation occurs, the amount of deformation is preferably not greater than 203.5 μm, and at least 1 μm, such as 1 μm, 25 μm, 50 μm, 75 μm, 100 μm, 125 μm, 150 μm, 175 μm, or 200 μm. The deformation amount can be defined as a distance between an apex of the protrusion of the magnetically-driven porous membrane 400 and its original position.

In summary, the present invention, by utilizing the magnetically-driven porous membrane 400 and the magnetic field generating module 60, provides a means that is completely different from traditional vacuum approaches. This method is able to simulate the dynamic microenvironment in the organism and the performances of organs, tissues, or cells in that dynamic microenvironment. Users can set up the power supply 70 and/or control module from the outside of the organ chip 10 to achieve the effect of simulating the dynamic microenvironment and the performances of organs, tissues, or cells in the dynamic microenvironment, which is more convenient compared to traditional methods that rely on vacuum system for gas extraction and delivery. In addition, unlike traditional vacuum system that require channels for gas extraction and delivery to be arranged inside the organ chip, the organ chip 10 of the present invention can have a simplified structure and a streamlined process thereof due to no requirement of the vacuum system, which is beneficial for cost down and yield improving.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A bionic organ device, comprising an organ chip and a magnetic field generating module, the organ chip comprising a first body, a second body, and a magnetically-driven porous membrane, wherein:
the magnetically-driven porous membrane is disposed between the first body and the second body and forms a channel system with the first body and the second body, the magnetically-driven porous membrane comprises a magnetic material and a membrane body, and the magnetic material is disposed in the membrane body; and
the magnetic field generating module is disposed outside the organ chip and is adapted to generate a magnetic field, the magnetic field passes through the magnetically-driven porous membrane and causes the magnetically-driven porous membrane to stretch, deform, or a combination thereof in response to a magnetic force in the magnetic field.

2. The bionic organ device according to claim 1, further comprising a power supply, wherein the power supply is electrically connected to the magnetic field generating module and is adapted to supply power to the magnetic field generating module.

3. The bionic organ device according to claim 2, wherein the magnetic field generating module comprises an electromagnet, the power supply further supplies an electric current, and the electric current is adapted to cause the electromagnet to have a magnetic effect and generate a magnetic field.

4. The bionic organ device according to claim 3, wherein the electromagnet is adapted to generate a magnetic force of 0 Kg to 30 Kg and a driving voltage of 12 V to 110 V.

5. The bionic organ device according to claim 1, wherein the magnetic material comprises a plurality of particles of iron, cobalt, nickel, or alloys or compounds thereof, the membrane body is made of a hydrophilic polymer material, and the plurality of particles of the magnetic material is distributed in the membrane body.

6. The bionic organ device according to claim 1, wherein the magnetic material is a carbonyl magnetic iron powder, the membrane body is made from hydrogel, and a weight ratio of the carbonyl magnetic iron powder to the hydrogel is 0.5:1 to 4:1.

7. The bionic organ device according to claim 1, wherein the magnetic force in the magnetic field is adapted to attract the magnetic material to drive the magnetically-driven porous membrane to stretch, deform, or a combination thereof.

8. The bionic organ device according to claim 1, wherein the magnetic force in the magnetic field is adapted to repel the magnetic material to drive the magnetically-driven porous membrane to stretch, deform, or a combination thereof.

9. The bionic organ device according to claim 1, wherein the magnetic field generating module is further disposed on the first body or the second body of the organ chip.

10. The bionic organ device according to claim 1, wherein the channel system comprises a first channel and a second channel, the first channel is located between the first body and the magnetically-driven porous membrane, the second channel is located between the second body and the magnetically-driven porous membrane, and a stretching of the magnetically-driven porous membrane comprises stretching toward the first channel or toward the second channel.

11. The bionic organ device according to claim 10, wherein the stretching of the magnetically-driven porous membrane toward the first channel compresses the first channel, and the stretching toward the second channel compresses the second channel.
